(19) United States
(12) Patent Application Publication
(10) Pub. No.: US 2005/0048513 A1
(43) Pub. Date: Mar. 3, 2005

(54) Rapid hybridization based on cyclical electric fields

(76) Inventors: Alex Harwit, San Mateo, CA (US); Junhong Wang, Foster City, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 10/695,685

(22) Filed: Oct. 28, 2003

Related U.S. Application Data
(60) Provisional application No. 60/499,311, filed on Aug. 28, 2003.

(51) Int. Cl. C12Q 1/68; G01N 33/53; C12M 1/34

(52) U.S. Cl. 435/6; 435/7.1; 435/287.2

(57) ABSTRACT

An apparatus for rapid hybridization of molecules and its methods of use is provided. The apparatus generates cyclical electric fields for electrically moving molecules to specifically enhance the binding efficiency of the molecules with other molecules. The apparatus includes an electrode pair that is in direct contact with a buffer, which includes the molecules. The electrodes and/or a lid could be used to vent generated gases. A temperature controlling means could be used to control the temperature of the buffer. The apparatus is amenable to use with a wide variety of microarrays making it cost-effective.

### CROSS-REFERENCE TO RELATED APPLICATIONS

This application is cross-referenced to and claims priority from U.S. Provisional Application 60/499,311 filed on Aug. 28, 2003, which is hereby incorporated by reference.

### FIELD OF THE INVENTION

The present invention relates generally to hybridization systems. More particularly, the present invention relates to a rapid hybridization apparatus and method.

### BACKGROUND

The age of genomics has presented us with a vast array of genomic sequences from many organisms (e.g. humans, mice, Drosophila, C. elegans, Arabidopsis and other animals and plants). The human genome project alone has identified approximately 3 billion base pairs of raw DNA sequences. This genomic data needs to be processed. The ability to derive as much information about as many genes as possible in the shortest time is of great importance. It is also equally important to correlate the genomic data with functional information. With the advent of microarray technologies, a researcher can now investigate over 30,000 genes simultaneously and compare expression patterns between normal and diseased states. Microarrays thus, present a methodology that can identify genes or pathways for new and unique potential drug targets, gene expression analysis, and mutation analysis. Microarray technology has become an important tool in large-scale genomics and proteomics analysis.

Microarrays were pioneered in the mid-1990's (Schena et al., Quantitative monitoring of gene expression patterns with a complementary DNA microarray, 1995, Science 270 (5235):467-70). They include thousands of discrete genes or probes printed as individual spots on a substrate, typically a glass microscope slide. The probes are exposed to mobile target molecules tagged with reporter molecules such as fluorescent dyes, suspended in a buffer solution. Hybridization between probe and target molecules occurs following DNA base-pairing rules. For example, targets may be DNA or mRNA samples prepared from two different cell populations. The two sample targets are first labeled using different fluorescent dyes. Common dyes include the red and green dyes Cy5 and Cy3, respectively. The targets are then hybridized to the microarray probes. After hybridization, a microarray scanner measures the fluorescent intensity from each spot for each dye tag. These measurements are used to determine the intensity of each dye signal and the ratio of, for example, red and green dyes, and in turn the relative abundance, of specific DNA target sequences in the two samples.

It is known in the art that nucleic acid hybridization can occur with both the probe and target in solution (Waittre P. Molecular Biology at the service of daily medical Virology 1. Daily Methodological Principles, 1997, Ann. Biol. Clin. 55(1):25-31) or with either probe or target fixed to a substrate, solid support hybridization (Southern E M. Detection of specific sequences among DNA fragments separated by gel electrophoresis, 1975, J. Mol. Biol. 98(3):503-517). Variations of solid support hybridization include Dot blot hybridization, Colony hybridization, and Sandwich hybridization. Dot blot hybridization involves covalently attaching the DNA to a filter and hybridizing it with a radiolabelled probe (Kafatos et al., Determination of Nucleic acid sequence homologies and relative concentrations by dot hybridization procedures, 1979, Nucleic Acid Res. 24;7(6):1541-1542). Dot blot hybridization can be varied to study gene mutation analysis and for the construction of genomic maps (U.S. Pat. No. 5,219,726). Colony hybridization involves affixing whole microorganisms typically to a nitrocellulose membrane filter, followed by denaturing of the DNA which remains affixed to the membrane and exposure to a radiolabelled oligonucleotide probe (Hogness D S. Colony hybridization: a method for the isolation of cloned DNAs that contain a specific gene, 1975, Proc. Nat. Acad. Sci. 72(10):3961-3965). Sandwich hybridization uses oligonucleotide probes covalently attached to a solid support as a bait to capture target nucleic acids (U.S. Pat. No. 4,563,419; Connor et al., Detection of sickle cell beta S-globin allele by hybridization with synthetic oligonucleotides, 1983, Proc. Natl. Acad. Sci. 80(1):278-282). However, these hybridization technologies have a number of limitations.

An example of a limitation of the existing hybridization technology is that non-specific base pairing occurs with non-target nucleic acids. It is known in the art that specificity can be increased by carrying out the hybridization reaction under high stringency conditions. The high stringency conditions are usually achieved by varying the concentration of salt, detergent, or denaturants, or by the addition of destabilizing agents or by changing the temperature of the hybridization mixture. The effects of these factors are known in the art and discussed in references, for example, Sambrook et al. Molecular Cloning: A Laboratory Manual, 2nd ed., vols. 1-3, Cold Spring Harbor Laboratory, 1989. Increasing the stringency may increase the specificity but it also results in a concomitant decrease in sensitivity.

Another example of a limitation is the ability to detect low copy numbers of a target nucleic acid and this remains a challenge with the existing hybridization technologies. Therefore, existing hybridization technologies result in a low sensitivity.

Yet another example of a limitation is the inefficiency of the existing hybridization technologies. Although it is possible to screen a large number of samples the process is time consuming. For example, the present state of the art enables one to use microarrays to screen the entire human genome in several weeks but lengthy hybridization times do not permit this to become reality. In an emergency situation such as SARS or bioterrorism a rapid hybridization test would be of immense value. Lengthy hybridization times are a limiting factor for achieving high throughput microarray hybridization systems.

Still another example of a limitation of existing microarray hybridization technologies is that compatibility of the hybridization chambers is limited to the manufacturer's microarray. This leads to a substantial investment of capital.

Accordingly, there is a need to develop a more universal hybridization apparatus that increases specificity of the probe-target interaction, detects very low levels of target molecules, is fast, cost-effective, and permits the end user to employ the apparatus with a large variety of existing microarray platforms.

### SUMMARY OF THE INVENTION

The present invention provides an apparatus and method for rapid hybridization by generating cyclical electric fields to electrically move and mix a first and a second molecule within a sample or reaction mixture. The use of cyclical electric fields to electrically move the molecules, rather than the buffer, increases the rate of binding of a molecule with its interacting partner, and decreases the time required for this event. The cyclical electric field also decreases non-specific interaction, thus increasing specificity.

The rapid hybridization apparatus of the present invention could be used in a wide variety of reactions, such as different microarray platforms and reaction mixtures and purification, and concentration of molecules. The apparatus includes a chamber filled with a buffer, which includes the microarray or the reaction mixture. Two electrodes are present on either side of the chamber and in direct contact with the buffer. A cyclical electric field generator establishes the cyclical electric field between the two electrodes and across (i.e. horizontally) the buffer to move the molecules in the reaction in a cyclical pattern.

The rapid hybridization apparatus of the present invention uses cyclical electric fields which can be used to not only hybridize interacting molecules, but also to purify and concentrate specific interactors, and/or to separate molecules. The apparatus can be used to hybridize a variety of molecules such as, for example, nucleic acids, polymers, peptides, proteins, antibodies, antigens, and tissues. The apparatus finds use in clinical assays, diagnostics, high through-put screening for genomics, biochemical separation, molecular genetic analysis, and nucleic acid diagnosis.

### BRIEF DESCRIPTION OF THE FIGURES

The objectives and advantages of the present invention will be understood by reading the following detailed description in conjunction with the drawings, in which:

FIG. 1 shows a side view and a top view of an example of a rapid hybridization apparatus with a reaction mix according to the present invention;

FIG. 2 shows a side view and a top view of an example of a rapid hybridization apparatus with any microarray according to the present invention;

FIG. 3 shows a scatter plot of rapid hybridization of Jurkat (Green) cDNA vs. Jurkat (Red) cDNA. Inset shows a subset of the array;

FIG. 4 shows two plots (4A and 4B) of fluorescent signal intensity versus cyclical frequency; and

FIG. 5 shows a comparison of the results of hybridization using cyclical electric fields (5A) versus hybridization using a state of the art technique (5B). A bar graph plotting the log2 ratio of data obtained from the Cy5/Cy3 channel is shown in 5C.

### DETAILED DESCRIPTION OF THE INVENTION

Although the following detailed description contains many specifics for the purposes of illustration, anyone of ordinary skill in the art will readily appreciate that many variations and alterations to the following exemplary details are within the scope of the invention. Accordingly, the following preferred embodiment of the invention is set forth without any loss of generality to, and without imposing limitations upon, the claimed invention.

The present invention is an apparatus for rapid hybridization, which could be used with a wide variety of reaction mixtures including those containing microarrays. The apparatus establishes cyclical electrical fields to move and hybridize molecules present in the buffer in an essentially cyclical pattern. The apparatus for rapid hybridization is also referred to as an electronic molecular mixing apparatus.

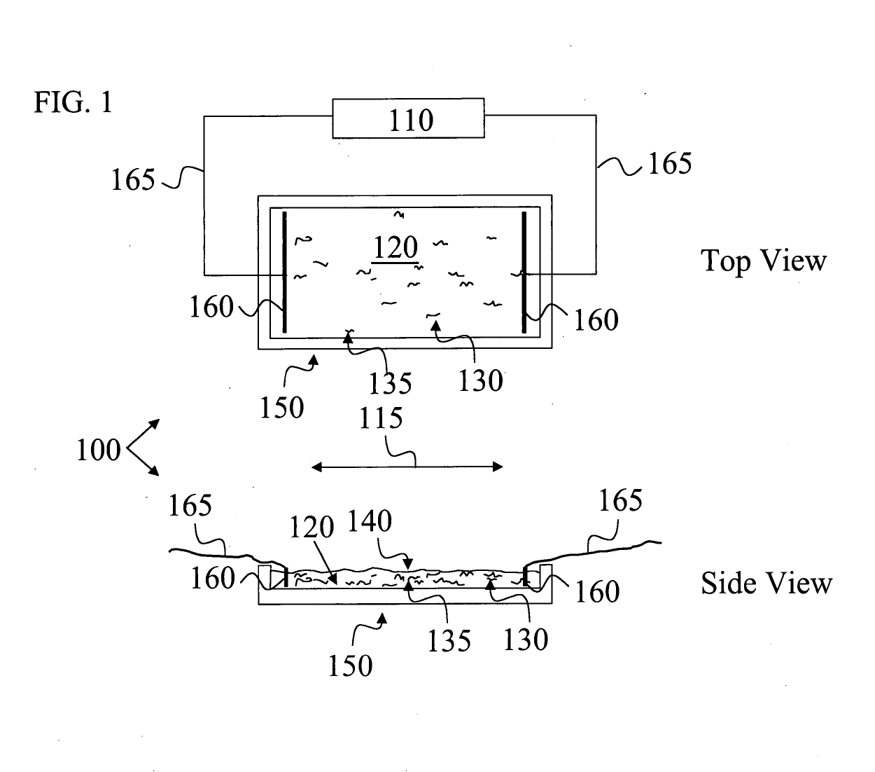

FIG. 1 shows an exemplary embodiment of a rapid hybridization apparatus 100. The apparatus 100 has a chamber 150, which includes a buffer 140, a first molecule 130, and a second molecule 135. The apparatus 100 includes two electrodes 160 in direct contact with the buffer. In one embodiment the electrodes have openings to vent generated gases. A cyclical electric field generator 110 establishes a cyclical electric field between the two electrodes 160 and across 115 the buffer 140 to cyclically and electrically move the first molecule 130 onto the second molecule 135. The cyclical electric field is applied in a more or less horizontal direction 115 across the buffer 140 to promote movement of the first molecule 130 across the second molecule 135, and thereby increase the probability of the first molecule 130 binding with the second molecule 135.

Hybridization occurs when a probe molecule binds to its target molecule. The first or the second molecule could either be the probe molecule or the target molecule. For example, in one embodiment of the invention, the first molecule is the probe molecule and the second molecule the target molecule. In another embodiment of the invention, the first molecule is the target molecule and the second molecule is the probe molecule. Probes and target molecules include, and are not limited to, nucleic acids, proteins, polymers, peptides, antibodies, antigens, and tissues. Probe and target molecules may be tagged or untagged. For example, a probe or target molecule may be tagged with a fluorescent dye to facilitate detection. In another example, a probe or target molecule may be tagged to facilitate its isolation after hybridization. The apparatus is capable of hybridizing a variety of molecules such as, for example, nucleic acids, polymers, peptides, proteins, antibodies, antigens, and tissues.

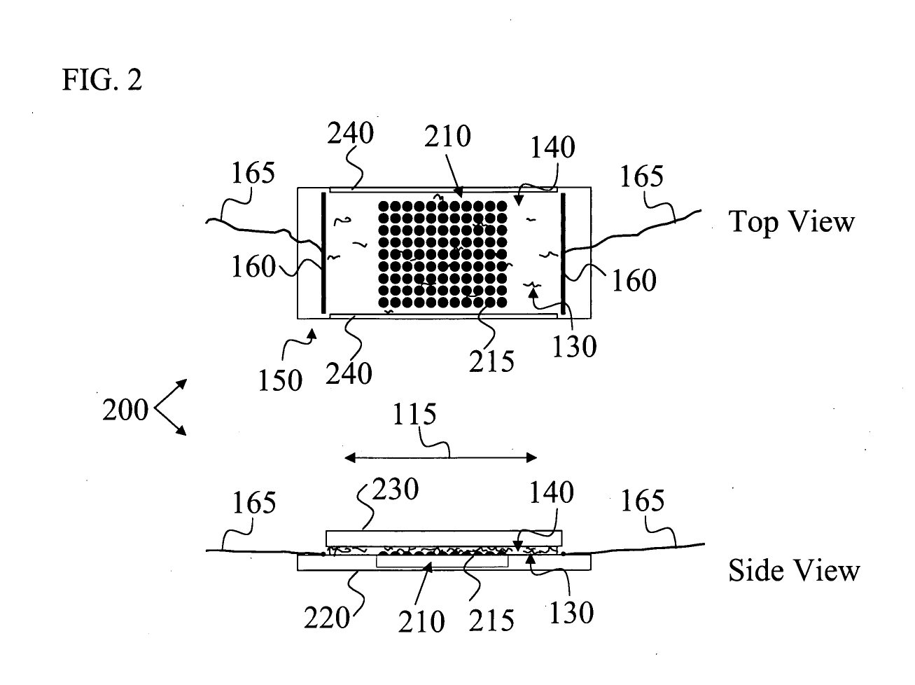

The hybridization could also occur between two mobile molecules or between a mobile and immobile molecule. In one example, both the first molecule and the second molecule are mobile and part of a reaction mixture 120, which is included in the buffer. In this example, hybridization occurs in the reaction mixture, or in solution. In another example, hybridization occurs between a mobile first molecule, which is part of a reaction mixture, and an immobile second molecule 215, which is part of a microarray 210 of FIG. 2 (with spots of the immobile second molecule 215 and base 220). In this example, hybridization occurs on the microarray, or on a solid support. The microarray may be, and is not limited to, a nucleic acid, a peptide, or a protein or tissue array.

In another example, the reaction mixture includes an immobile probe molecule, which is part of a microarray and a mobile target molecule suspended within a buffer. In yet another example, the reaction mixture could include an immobile target molecule with a mobile probe molecule suspended within a buffer. In still another example, the reaction mixture could include a mobile target molecule with a mobile probe molecule suspended within a buffer. After binding, probe-target hybrids may be detected by a variety of methods known in the art, for example, fluorescence, chemiluminescence, and electrochemiluminescence.

The apparatus has two electrodes 160 which are in direct contact with the buffer solution 140 and carry the current generated by the cyclical electric field generator 110. The two electrodes are spaced to include the reaction mixture or the microarray. For example, the electrodes may be placed either at the edge of the buffer solution or near the edge of the buffer solution as long as the molecules are situated inside the space between the two electrodes. The electrodes are in electrical contact by means of wires 165 with cyclical electric field generator 110. In one embodiment, the electrodes have holes or are part of an electrode with gaps to vent any gases generated during the hybridization process. For example, the electrodes may have holes or be part of an electrode array with gaps between the electrodes. The electrodes could be wire electrodes or made of any other conductive material. For example, the two electrodes may be made of silver, silver alloy, gold, gold alloy, copper, copper alloy, glassy carbon or a conductive gel. The conductive gel may have dual functionality. In addition to conducting the current, conductive gel may also be used to seal the apparatus and minimize evaporation. In another embodiment of the invention, a lid 230 with a small hole or a plurality of holes may be used to vent gases generated by the rapid hybridization apparatus while minimizing the effects of evaporation. The lid may in certain embodiments not extend past the electrodes to vent gases generated by the rapid hybridization apparatus while minimizing the effects of evaporation. The apparatus may also be tilted for this purpose to allow bubbles to accumulate under and/or vent through the holes during the hybridization process. If tilted, the electrical field across the buffer and between the electrodes is still established in the horizontal direction. The horizontal direction is then defined in line with the rotation of the apparatus. The lid and spacer 240 could be combined together, which is also known in the art as a LifterSlip™. In yet another example, the enclosed space could have small holes machined into lid 230 to vent gases generated by the rapid hybridization apparatus 200.

The composition of the buffer 140 will depend on the nature of the molecules to be hybridized, and the concentration and complexity of the molecules. The chamber 150 of the apparatus could be filled with a buffer solution 140, which includes the reaction mixture 120, the first molecule 130, and the second molecule 135. The second molecule may be part of a microarray 210. In one example, the buffer could substantially fill the volume of the chamber. In another example, the buffer could cover just the base of the chamber. The resistivity of the buffer may in some cases be low, typically on the order of 25 Ohm-cm. The resistivity of the buffer can be manipulated, for example, by varying the salt concentration. In addition, the buffer may contain any combination of conductive elements, denaturants, detergents, salts, etc.

The cyclical electrical field generator electrically moves and mixes the molecules in a cyclical pattern. The cyclical pattern includes, and is not limited to, square, sinusoidal, or triangular waveforms. The cyclical patterns may be used in combination with possibly zeroing the polarity between the cyclical patterns. Moving the molecules selectively, rather than the buffer, will increase the chances of interacting molecules to find each other and bind. This results in a shorter hybridization time and increased sensitivity. The cyclical electric field may influence the reaction in other ways. For example, the cyclical electric field may “stretch” or unwind nucleic acids exposing more binding sites and permitting more probe-target interactions, thus increasing sensitivity.

The voltage range is selected from a range of 1-340 Volts. In one aspect, the electrodes could have a spacing of 60 mm and the cyclical electric field may be selected from a range of 0.17-56.7 Volts/cm. In the above-mentioned embodiments, the cyclical frequency is selected from a range of approximately 2-940 Hertz. These embodiments may include a frequency divide circuit with taps spaced at factors of two and within a controller. By changing a single connection, the frequency can be reduced by any of 9 taps or by a factor of 2, 4, 8, 16, 32, 64, 128, 256, or 512. For example, by switching to the divide by 32 tap, the output frequency range would be 1/32 of its present value or approximately 0.06 to 29.4 Hz. In principle, the output of one controller can be time synchronized with another controller and the controller outputs combined in series to produce double the voltage. Two controllers connected in this way could produce voltages in the range of 2 to 680 volts. This mode of operation may apply to more than 2 controllers for even higher output voltages. An example of an advantage of such a system, would be to mitigate some of the controller limitations related to simultaneous high voltages and high frequencies. It will also be appreciated by those skilled in the art that the voltage selected is dependent on a number of factors, for example, the resistivity of the buffer, the nature of the molecules being hybridized, and the complexity of the probe and target pool. These parameters will determine the voltage, frequency, and strength of the cyclical electric field used in each reaction. For example, the cyclical electric field generator could include one or more of the following devices such as, for example, an adjustable frequency oscillator, a high voltage amplifier, an adjustable voltage power supply, a fixed voltage power supply, an optoisolator, an optocoupler, a photocoupler, or a solid-state relay.

The cyclical electric field could increase the temperature of the buffer. In addition, the reaction could cause an increase in the temperature. In some cases, it is necessary to keep the temperature controlled which could be accomplished by including a temperature controlling means. For example, the temperature controlling means could be a thermoelectric cooler, a water pack, an ice pack, a piece of metal, a fan, a heater, or a dispenser that replenishes evaporated buffer.

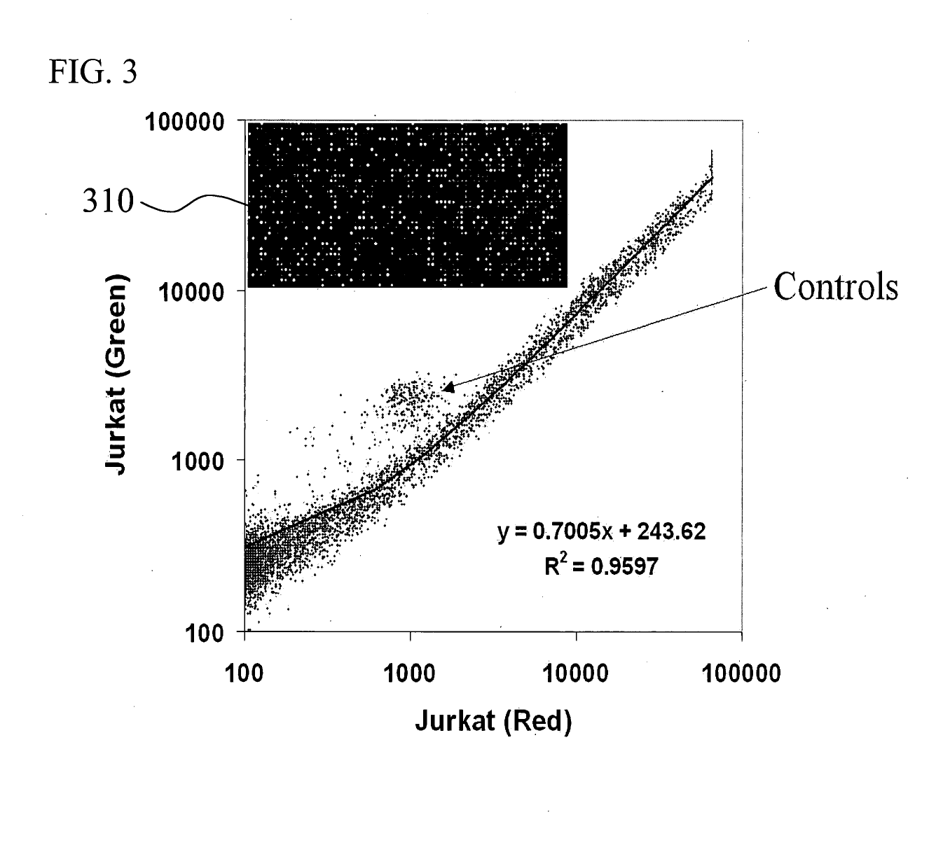

FIG. 3 shows exemplary results obtained with Agilent Human 1A Oligo microarrays. The targets were Jurkat cDNA labeled with Cy3 and with Cy5 (self vs. self) plus Agilent recommended control target. FIG. 3 (inset 310) shows a subset of the microarray after a 20-minute hybridization in a cyclical electric field. Hybridization was performed at room temperature using a cyclical voltage of 79-83 volts at a frequency of 16.4 Hz. The current was about 5 mA. The hybridization cell utilized a LifterSlip™ (Erie Scientific Company, catalog number 25×60I-M-5439) and the electrodes were separated by 60 mm. The 100 microliter of hybridization mix contains 80 microliters of WH1 Buffer and 20 microliters of labeled targets in deionized water. After hybridization, the microarray slides were first washed with 2×SSC (Saline-Sodium Citrate buffer) plus 0.1% SDS (Sodium Dodecyl (Lauryl) Sulfate), then washed with 0.1×SSC plus 0.1% SDS, the last wash was with 0.1×SSC. Each one (1) minute wash was done at room temperature. The buffer WH1 is composed of 4×SSC (diluted from Ambion Cat #9763), 0.1% SDS (diluted from Ambion Cat #9820), 15 ug ssDNA (Invitrogen, Cat#15632011), 3.5M Urea (Sigma Cat#5378), 10 mM L-Histidine (Sigma Cat #53320), and 1 uM CTAB (Hexadecyltrimethyl-Arnmonium Bromide, Sigma Cat# H6269). FIG. 3 shows a scatter plot of the data. Data obtained from the Agilent controls, which includes spiked-in positive, prelabeled controls, confirm that the hybridization worked as expected. Note that the dye used for these control targets overlaps both the Cy3 and Cy5 channels. The scatter plot shows that the intercept for the data crosses the Y-axis above zero. This indicates that the green Jurkat target was more strongly labeled than its red counterpart. Regression analysis shows a 0.96 correlation between the two samples.

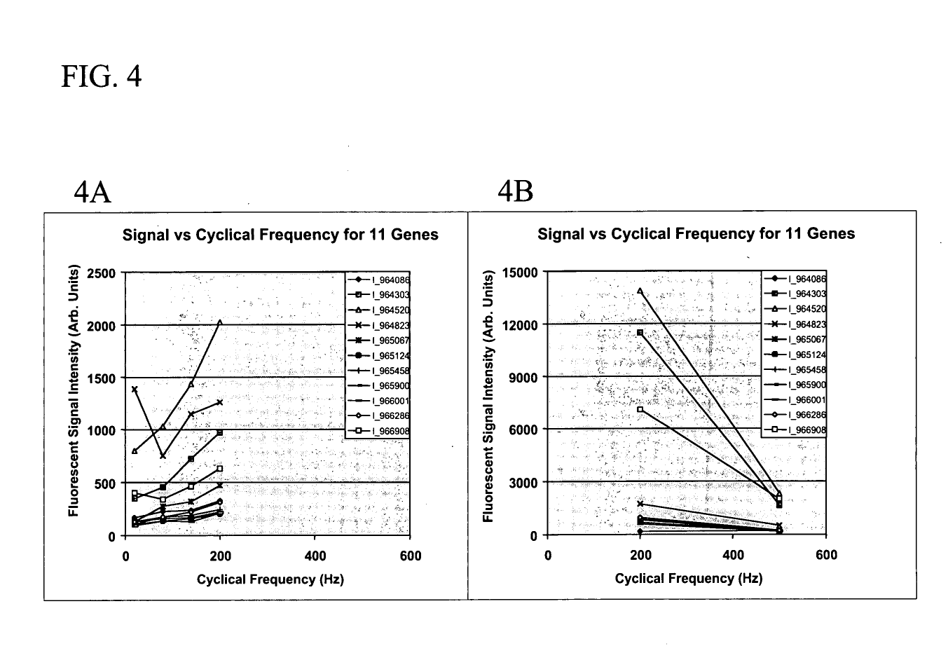

FIG. 4A and FIG. 4B show the results of varying the cyclical frequency over the range of 20 to 500 Hz. The results are presented for 11 representative genes. The conditions are similar to those for the results of FIG. 3. The temperature controlling means is a piece of metal. The voltage is in the range 88 to 115V and the current approximately 5 mA (a quasi-constant current mode). In this example, the results indicate that under these conditions, a maximum signal intensity is obtained at a frequency of 200 Hz and an acceptable signal intensity is obtained between the frequencies of 140 and 500 Hz. It is expected that the frequency at which the maximum signal intensity and the acceptable signal intensity is obtained will vary depending on the environmental conditions and on the molecules being hybridized.

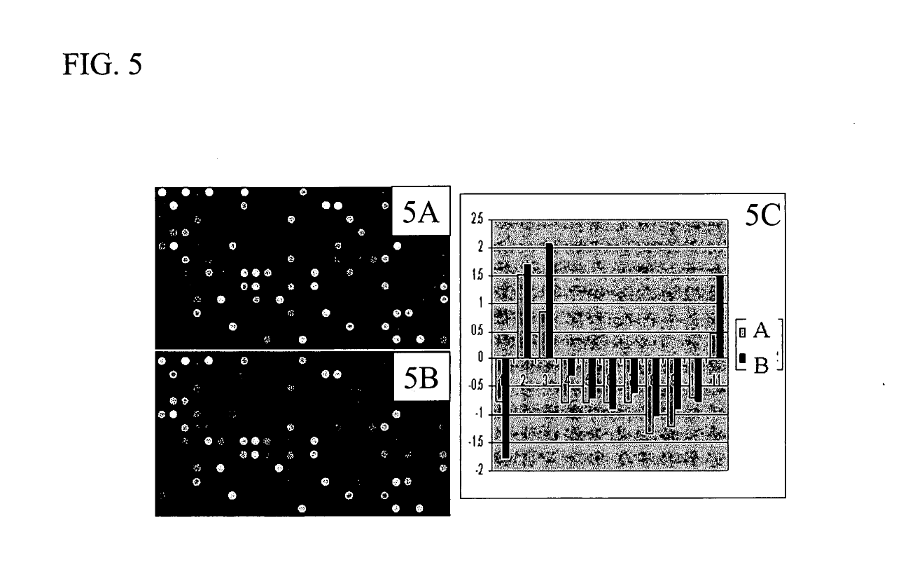

FIG. 5 shows a specificity profile obtained by comparing the hybridization results obtained with the apparatus for rapid hybridization using cyclical electric fields FIG. SA versus hybridization with a state of the art 20-hour hybridization apparatus FIG. 5B. In FIG. 5A an Agilent Human 1A Oligo microarray is hybridized using cyclical electric fields at a frequency of 200 Hz for 15 minutes using WH1 buffer at room temperature. The voltage is approximately 94.5±2 volts, while the constant current mode hybridization is maintained at 4.7 mA. FIG. 5B shows an Agilent Human 1A Oligo microarray hybridized using Agilent's standard protocol at 60° C. for 20 hours. The target was prepared and split into two to perform the two hybridizations. FIG. 5C shows a bar graph which plots the Log2 ratio of Cy5/Cy3 of 11 genes hybridized under the two different conditions. Images of the same regions of the two microarrays show good correlation as is confirmed in the specificity profile FIG. 5C.

The present invention has now been described in accordance with several exemplary embodiments, which are intended to be illustrative in all aspects, rather than restrictive. Therefore, it will be appreciated by those skilled in the art that the above embodiments may be altered in many ways without departing from the scope of the invention. For example, in one aspect, cyclical electric fields can be used to rapidly purify DNA or RNA according to the following steps: a) Forming a layer of immobile first molecules on a substrate or a microarray; b) Adding the second molecule to the buffer; c) Using the rapid hybridization apparatus, and binding the second molecule onto the layer of the first molecules on the substrate or on a microarray; d) Removing the microarray or substrate containing the hybridized molecules; e) Denaturing and harvesting the second molecule from the microarray or substrate containing the hybridized molecules and; f) Placing the microarray or the layer of immobile first molecules on a substrate back into the buffer containing the second molecule; g) Repeating the steps “c” through “f” to hybridize and harvest more of the second molecule.

In another aspect of the invention, the apparatus for rapid hybridization using cyclical electric fields may be used, for example, to dissociate or to unbind the target from the probe in a pre-hybridized microarray. The pre-hybridized microarray with a target already bound to a probe may be obtained, for example, by methods known in the art, or by using the methods and apparatus of the present invention. In this example, the pre-hybridized microarray was prepared using methods known in the art, and was placed in the apparatus for rapid hybridization, without a lid. The target was dissociated from the probe using the following conditions: a buffer composed of 10 mM Histidine, 0.1% SDS and 0.5 uM CTAB; applying a voltage approximately 60 Volts (±60 Volts); applying a frequency of approximately 0.1 Hz and; a current of 3 to 4 mA. The cyclical electric field was applied for 10 minutes. The buffer covered only a section of the pre-hybidized microarray and hence only a section of the pre-hybridized microarray received the cyclical electric field. The section that did not receive the cyclical electric field is referred to as the control section. Subsequent to the application of the cyclical electric field, the entire pre-hybridized microarray was washed with a median and high stringency washing buffer. The pre-hybridized-microarray was then scanned at the same PMT (Photomultiplier Tube) settings used before the dissociation. Comparisons were made between the sections of the pre-hybridized microarray that received the cyclical electric fields versus the control section, and between the same microarray spots before and after they received the cyclical electric fields. In each case, the intensities of many spots were measured. Results showed that on average, the signal intensity decreased by a factor of 18, when comparing the intensities of the spots before, and after, receiving the cyclical electric fields. Results also showed that when the intensities between 8 control spots and the 8 spots that received the electric fields was compared, the decrease was by a factor of about 8. In this example, the cyclical electric fields are used to dissociate the target from the pre-hybridized microarray and move the target into the buffer. It will be appreciated by those skilled in the art, that the apparatus for rapid hybridization may be used, for example, in other methods, such as purification of molecules where they may replace the step of denaturation.

In another aspect, the use of the present invention is not limited to electrically mixing nucleic acids. It is possible to use the present invention to purify proteins or any charged biologically relevant material with a certain specific affinity or capability to form separable complexes.

In yet another aspect of the invention, the cyclical frequency is tuned to the resonance frequency of a first or second molecule. This can be used to either enhance binding of a first or second molecule or to retard binding of a first or second molecule within a reaction mixture.

In yet another aspect of the invention, one could inject a first molecule or a second molecule though a cell wall and/or plasma membrane into a cell resulting in the molecule being transferred from outside the cell into the cell.

In yet another aspect of the invention, the cyclical electric field generated by the apparatus is used to inject a nucleic acid through a cell wall and/or plasma membrane into a cell and functionally modify a cell. For example, one could replace nucleic acids with proteins or charged biological material or a carrier with a drug attached.

In still another aspect of the invention, the cyclical voltage may be changed to a different cyclical voltage, for example, to increase the specificity of the hybridization or to decrease the hybridization time. In still another aspect of the invention, the cyclical electric field may be changed to a different cyclical electric field, for example, to increase the specificity of the hybridization or to decrease the hybridization time. The cyclical frequency may also be changed to a different cyclical frequency, for example, to increase the specificity of the hybridization or to decrease the hybridization time. The temperature may also be changed to a different temperature, for example, to increase the specificity of the hybridization or to decrease the hybridization time.

The rapid hybridization apparatus and method of the present invention using cyclical electric fields does not require expensive capital equipment. Furthermore, the apparatus and method is compatible with the majority, if not all, existing microarray platforms. The present invention has applications in medical research, in hospitals for rapid diagnosis and treatment of diseases and in the field for assessment of biological hazards such as bio-terrorism. More specific applications include, and are not limited to, measuring DNA and RNA levels, genotyping and gene identification, tissue studies, identifying protein binding sites, and studying immunologic responses to infections and biological agents.

All such variations and other variations are considered to be within the scope and spirit of the present invention as defined by the following claims and their legal equivalents.

1. An apparatus for rapid hybridization, comprising:
(a) a chamber having a buffer, a first molecule, and a second molecule;
(b) two electrodes, spaced on either side of said chamber, and in direct contact with said buffer; and
(c) a cyclical electric field generator to establish a cyclical electric field between said two electrodes to electrically move said first molecule across said buffer in a cyclical pattern to bind said first molecule with said second molecule

2. The apparatus of claim 1, wherein said first molecule is a mobile molecule.

3. The apparatus of claim 1, wherein said second molecule is a mobile molecule or an immobile molecule.

4. The apparatus of claim 1, wherein said second molecule is part of a microarray.

5. The apparatus of claim 1, wherein said first molecule or said second molecule is a nucleic acid, a protein, a polymer, a peptide, an antibody, an antigen, or a tissue.

6. The apparatus of claim 1, wherein said electrodes have holes or provide a gap to vent generated gases.

7. The apparatus of claim 1, wherein said chamber further comprises a lid.

8. The apparatus of claim 1, wherein said cyclical electric field generator generates a cyclical voltage selected from a range of 1-680 Volts.

9. The apparatus of claim 1, wherein said cyclical electric field generator generates a cyclical electric field selected from a range of 0.17-113 Volts/cm.

10. The apparatus of claim 1, wherein said cyclical electric field generator generates a cyclical frequency selected from a range of 0.06-940 Hertz.

11. The apparatus of claim 1, wherein said cyclical electric field generator comprises of an adjustable frequency oscillator, an adjustable voltage power supply, a fixed voltage power supply, a high voltage amplifier, a solid state relay an optoisolator, an optocoupler, or a photocoupler.

12. The apparatus of claim 1, further comprising a temperature controlling means for controlling the temperature of said buffer.

13. The apparatus of claim 1, wherein said cyclical pattern is a square, a triangular, a sinusoidal or a step pattern.

14. A method of rapid hybridization of molecules comprising the steps of:
a. providing a chamber with a buffer, a first molecule and a second molecule;
b. providing two electrodes on either side of said chamber and in direct contact with said buffer; and
c. generating a cyclical electric field between said two electrodes to electrically move said first molecule across said buffer in a cyclical pattern to bind said first molecule with said second molecule.

15. The method of claim 14, wherein said cyclical electric field increases the binding of said first molecule to said second molecule.

16. The method of claim 14, wherein said cyclical electric field decreases the binding of said first molecule to said second molecule.

17. The method of claim 14, wherein said cyclical electric field injects said first molecule or said second molecule through a cell wall into a cell.

18. The method of claim 14, wherein said cyclical electric field has a cyclical voltage selected from a range of 1-680 Volts.

19. The method of claim 18, further comprising the step of changing said cyclical voltage to a different cyclical voltage from said cyclical voltage, wherein said different cyclical voltage is selected from a range of 1-680 Volts.

20. The method of claim 14, wherein said cyclical electric field is selected from a range of 0.17-113 Volts/cm.

21. The method of claim 20, further comprising the step of changing said cyclical electric field to a different cyclical electric field from said cyclical electric field, wherein said different cyclical electric field is selected from a range of 0.17-113 Volts/cm.

22. The method of claim 14, wherein said cyclical electric field has a cyclical frequency selected from a range of 0.06-940 Hertz.

23. The method of claim 22, further comprising the step of changing said cyclical frequency to a different cyclical frequency from said cyclical frequency, wherein said different cyclical frequency is selected from a range of 0.06-940 Hertz.

24. The method of claim 14, wherein said first molecule is a mobile molecule.

25. The method of claim 14, wherein said second molecule is a mobile or an immobile molecule.

26. The method of claim 14, wherein said cyclical electric field denatures said first or said second molecule.

27. The method of claim 14, wherein said first molecule or said second molecule is a nucleic acid, a protein, a polymer, a peptide, an antibody, an antigen, or a tissue.

28. The method of claim 14, wherein said second molecule is part of a microarray.

29. The method of claim 14, further comprising the step of controlling the temperature of said buffer.

30. A method of rapid hybridization of molecules comprising the steps of:
(a) providing a chamber having a buffer and two electrodes in direct contact with said buffer and positioned on either side of said chamber;
(b) forming a layer of immobile first molecules on a substrate and placing said layer of immobile first molecules in said chamber;
(c) adding a second mobile molecule in said buffer;
(d) establishing a cyclical electric field between said two electrodes and across said buffer;
(e) electrically moving said second molecule, across said buffer, in a cyclical pattern, binding said second mobile molecule with said layer of immobile first molecules on a substrate, and forming a layer of hybridized first and second molecules on a substrate;
(f) removing the said layer of hybridized first and second molecules on a substrate;
(g) denaturing the said layer of hybridized first and second molecules on a substrate and forming a denatured second molecule(s), and the said layer of immobile first molecules on a substrate;
(h) harvesting said denatured second molecules;
(i) placing the said layer of immobile molecules on a substrate back into said buffer with said second molecule; and
(j) repeating the steps “d” through “i”.

31. A method of rapidly dissociating molecules comprising the steps of:
(a) providing a chamber having a buffer and two electrodes in direct contact with said buffer and positioned on either side of said chamber;
(b) forming in said chamber a layer of second molecules bound to a layer of immobile first molecules on a substrate, and placing said layer of second molecules bound to said layer of immobile first molecules on said substrate; and
(c) establishing a cyclical electric field between said two electrodes and across said buffer to electrically unbind said second molecule from said layer of second molecules bound to said layer of immobile first molecules on said substrate.
